(19) United States
(12) Patent Application Publication
(10) Pub. No.: US 2009/0247641 A1
(43) Pub. Date: Oct. 1, 2009

(54) HYDRAZONE AGENTS TO TREAT CUTANEOUS LESIONS

(76) Inventor: Lee Roy Morgan, New Orleans, LA (US)

Correspondence Address: DICKSTEIN SHAPIRO LLP, 1825 EYE STREET NW, Washington, DC, 20006-5403, US

(21) Appl. No.: 12/093,417

(22) PCT Filed: Aug. 25, 2006
(86) PCT No.: PCT/US06/33318

§ 371 Date: May 12, 2008

Related U.S. Application Data
(60) Provisional application No. 60/711,825, filed on Aug. 26, 2005.
(60) Provisional application No. 60/740,979, filed on Nov. 29, 2005.

(51) Int. Cl. A61K 31/15; A61P 35/00; A61P 17/12

(52) U.S. Cl. 514/639

(57) ABSTRACT

A method is disclosed for treating hyperproliferative body surface lesions, including cancerous or precancerous lesion, such as warts or anogenital cancers, by applying a polyaryl mononitro- or dinitrophenylhydrazone such as (I) wherein R¹ is hydrogen, hydroxy, 2- or 4-hydroxyphenyl, acetate, nitroso, phosphate, azido, nitrile, amino, dimethylamino, sulfate, methylsulfonate, phosphate, succinate or another water soluble electrophilic group capable of hydrogen bonding; R² is C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; and X is C₆H₃-2,4(NO₂)₂, C₆H₄-4(NO₂), C₆H₄-3(NO₂), or C₆H₃-2,4(NO₂)₂. In a particular example, R¹ is OH, R² is C₆H₄OH and X is C₆H₃-2,4(NO₂)₂.

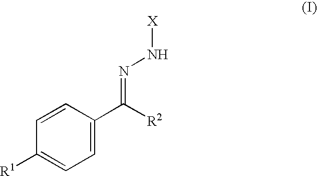

### CROSS REFERENCE TO RELATED APPLICATION

This application is a continuation-in-part (CIP) of U.S. patent application Ser. No. 10/547,223 filed Aug. 24, 2005, which is the national stage of PCT/US2004/005845 filed Feb. 27, 2004, and claims the benefit of U.S. Provisional Patent Application No. 60/450,877 filed Feb. 28, 2003. This application also claims the benefit of the earlier filing date of U.S. Provisional Patent Application Nos. 60/711,825 and 60/740,979, filed Aug. 26, 2005 and Nov. 29, 2005, respectively. Each of these prior applications is incorporated herein by reference.

### STATEMENT OF GOVERNMENT SUPPORT

The U.S. Government may have certain rights in the invention. Some work related to the development of this invention was carried out under National Cancer Institute Grant Number R43 CA89772-01.

### FIELD

This invention concerns the treatment of skin lesions, such as hyperproliferative lesions associated with infection or inflammation. In particular examples, the lesion is a cancerous or precancerous lesion of the anus or genitals, such as cancer of the penis, vulva or vagina.

### BACKGROUND

Body surfaces function as an interface with the external world. The skin covers most of the external body surface and assists with thermoregulation and protection from the environment. Internal body surfaces also protect against environmental insults while serving other important biological functions. The cervical epithelium, for example, protects the cervix while performing reproductive functions. The anal epithelium is an important element of the digestive tract. The oral epithelium lines the oral cavity and comes into frequent contact with potential pathogens and inflammatory agents.

The protective function of these external and internal body surfaces brings these surfaces into contact with infectious and inflammatory agents that can harm them. The skin, for example, protects the body from ambient ultraviolet radiation by absorbing the radiation. This radiation in turn causes inflammation and genetic damage that over time induces hyperproliferative lesions such as benign and malignant skin cancers. Body surfaces are also an important barrier against infectious pathogens, such as viruses, bacteria and fungi. Although the body surfaces often protect against systemic infection with these agents, localized infection of skin and mucosal surfaces is a widespread problem. Chronic infections often lead to more serious problems, such as warts and tumors. An example of a particularly serious complication of such chronic infection is dysplastic or neoplastic transformation of surfaces, such as cervical epithelium, infected with the papillomavirus.

Infection of the anogenital skin with human papillomavirus (HPV) results in exophytic or flat warts, and infection with some HPV genotypes is recognized as a cause of anogenital cancer. Molluscum contagiosum is a poxvirus associated veneral wart that also infects these tissues and is resistant to treatment. Genital warts are often transmitted sexually, infecting the vulva, vaginal wall, cervix and areas around the vagina. In men, the penis and surrounding areas such as the perineum may be infected. The anus, rectum, mouth and throat are other sites of infection in both men and women.

Even those warts (verrucae) that do not lead to malignancy are a significant problem. Plantar warts develop on the sole of the foot, causing pain while walking and thickening of surrounding skin. Filiform warts form long, unsightly small growths on the eyelids, face, neck or lips. Flat warts appear as smooth yellow-brown spots on the face, and seborrheic warts are generally found on the faces of the aged. These warts are often unsightly and difficult to treat.

Penile cancer is in some instances believed to be related to HPV infection; however other cases are instead believed to be caused by poor hygiene, particularly in uncircumsized males.

Current treatments for genital and other types of warts are generally destructive and include the use of surgery, laser ablation, cryotherapy, or caustic chemicals. Treatments for anogenital cancers are primarily limited to surgical resection. Such surgery on the external genitalia, such as the penis or vulva, often cause severe disfigurement and psychological trauma.

Many other hyperproliferative lesions of the skin are known. They include conditions such as psoriasis in which increased epidermal cell proliferation is accompanied by dermal inflammation, and herpes simplex infection, which is associated with painful vesicular eruptions.

It would be helpful to have a convenient and effective treatment for hyperproliferative body surface lesions, including cancerous and precancerous lesions, such as skin cancers and warts.

### SUMMARY OF THE DISCLOSURE

Disclosed herein is a method of treating a hyperproliferative body surface lesion by applying to the lesion an effective amount of a compound

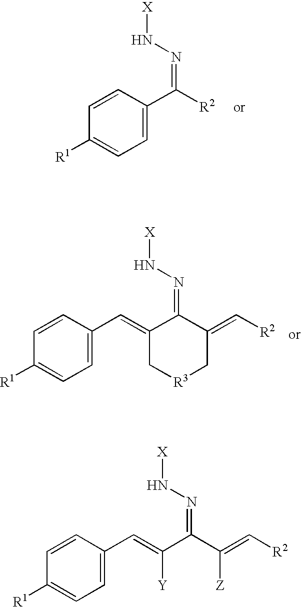

wherein R¹ is hydrogen, hydroxy, 2- or 4-hydroxyphenyl, acetate, phosphate, azido, nitrile, amino, dimethylamino, sulfate, methylsulfonate, phosphate, succinate;

R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;

X is C₆H₃-2,4(NO₂)₂, C₆H₄-4(NO₂), C₆H₄-3(NO₂), or C₆H₃-2,4(NO₂)₂;

R³=—O—, —S—, —CH₂—, —N—, —, —CHA- and —CHOA-; where A=aryl, ester, amide, lipid, carbohydrate, or peptide;

Y=H, (CH)ₓCH₃ (x=0-12), —S—CH₃, nitrile, amino, nitro, azido, succinate, or amide; and

Z=H, (CH)ₓCH₃ (x=0-12), —S—CH₃, nitrile, amino, nitro, azido, succinate, or amide.

In a particular example the compound is a 2,4-dinitrophenylhydrazone (referred to as A-007), wherein the compound is

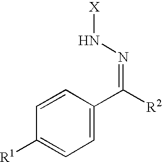

### and R¹ is OH, R² is C₆H₄OH and X is C₆H₃-2,4(NO₂)₂.

These compounds can be used to treat an internal or external body surface lesion. Such lesions can include an anogenital lesion, such as a genital or anal lesion, for example a wart or malignancy, such as a primary malignancy. In particular disclosed examples the lesion is an internal lesion, such as a genital wart of the female reproductive tract, such as a cervical wart. In one embodiment the lesion is a precancerous lesion. Indeed, in some examples the lesion is a papillomavirus associated lesion, such as an HPV-induced lesion. In yet other examples the lesion is a non-venereal wart, such as a plantar wart, filiform wart, flat wart or seborrheic wart. In certain embodiments, the disclosed compounds can be used to exert a prophylactic effect, such as by treating a precancerous lesion and preventing or delaying neoplasia of the tissue. The compounds are particularly suitable for treating genital lesions caused by a variety of pathologies, including cancers of the vagina and cancers of the external genitalia, such as primary cancers of the penis or vulva.

In particular examples, the compound is applied topically to the body surface lesion or introduced into the skin, for example by injection or placement of a pellet into the lesion. Alternatively the agent is applied in a pad to the lesion, and the pad is left in place over the lesion for an extended period of time, such as one or more days or a week or more.

In other examples, the lesion is a skin lesion induced by infection or inflammation. The lesion may be a virally induced lesion, such as a wart, for example a papillomavirus induced wart. Alternatively, the lesion being treated is a bacterial (for example staphylococcus) or viral (for example herpes virus) infection of the skin, or a skin neoplasm (for example a skin cancer such as a squamous or basal cell carcinoma or a primary melanoma).

The foregoing and other objects, features, and advantages of the method will become more apparent from the following detailed description of several embodiments which proceeds with reference to the accompanying figures.

### BRIEF DESCRIPTION OF THE FIGURES

FIG. 1 is a schematic illustration of the interaction between a 4,4′-dihydroxybenzophenone-2,4-dinitrophenylhydrazone (A-007) and the CD45+ T-lymphocyte surface receptor.

FIG. 2 illustrates the structural formula of A-007.

FIG. 3 is a schematic illustration of the resonance structures of A-007.

### DETAILED DESCRIPTION

### I. Abbreviations

A-007: 4,4′-dihydroxybenzophenone-2,4-dinitrophenylhydrazone

APC: Antigen Presenting Cell

DNP: Dinitrophenylhydrazone

HPV: Human Papillomavirus

PTP: Protein Tyrosine Phosphatase

### II. Terms

Unless otherwise noted, technical terms are used according to conventional usage. If any of the following terms conflict with a document that has been incorporated by reference, the meanings of terms set forth herein will control.

In order to facilitate review of the various embodiments of this disclosure, the following explanations of specific terms are provided:

Anogenital: Relating to the anus and genitals.

Body surface: The body surface includes both internal and external surfaces that serve as an interface with the environment outside the body, and can be treated with the method disclosed herein. The skin is the most prominent body surface, and it serves as a barrier between the ambient environment and the internal organs. The skin covers many important surfaces that can be infected, and may be the site of lesions such as warts. Examples include the skin of the face, hands, feet (particularly the soles of the feet), genitals and perineum. However the body surface also includes structures enclosed within the body that serve as interfaces with the environment, particularly the surfaces of the oral, anal and vaginal cavities. Each of these surfaces comes into contact with the environment although these surfaces are not readily visible to an observer. For example, the skin or mucosal surfaces of vaginal, oral and anal cavities are exposed to potential pathogens during sexual activity, and are the site of lesions that can be treated with the compounds disclosed herein.

Cancerous: Referring to a malignant tumor or lesion, such as a primary cancer of the penis, vulva, vagina, uterine cervix, or integument. An example of a cancer of the penis is an invasive squamous cell carcinoma. One example of cancer of the vagina is squamous cell carcinoma. Any of these tumors can be treated with the methods described herein.

Genital: Relating to the external sex organs, for example the penis and vulva.

Hydrazone: A compound with the structure R₂C═NNR₂, differing from a ketone or aldehyde by the replacement of the double bonded oxygen with the ═NNR₂. A hydrazone is generally formed by the condensation of a hydrazine with a carbonyl group. An aryl hydrazone is a hydrazone in which at least one of the R groups is an aryl group, for example a phenyl group (a phenylhydrazone). A nitrophenylhydrazone is a phenylhydrazone having one or more NO₂ substitutions on the phenyl ring.

Hyperproliferative: Proliferating in an abnormal manner. Examples of hyperproliferative lesions that can be treated with the compounds disclosed herein include psoriatic lesions, warts (including plantar warts and genital warts), HSV blisters, and skin cancers such as basal cell carcinoma, squamous cell carcinoma, and melanoma.

Infectious agent: An agent that can infect a subject, including, but not limited to, viruses, bacteria, and fungi.

Lesion: An abnormal change in a body part due to illness or injury. For example, a skin lesion can be a hyperproliferative growth such as a wart or psoriatic area, or a blister induced by an HSV infection.

Neoplasm: An abnormal cellular proliferation, which includes benign and malignant tumors, as well as other proliferative disorders.

Papillomavirus: Papillomaviruses are small, nonenveloped viruses with an icosahedral symmetry, capsomere, and a double-strand circular DNA genome of about 8,000 bp. All papillomaviruses have a similar genetic organization. The viral genome is divided into an early region which encodes the genes required for viral DNA replication and cellular transformation, a late region that codes for the capsid proteins, and a regulatory region that contains the origin of replication and many of the control elements for transcription and replication.

Papillomaviruses have a high degree of species specificity. There are no known examples of natural transmission of human papillomavirus (HPV) to other species. Papillomaviruses also display a marked degree of cellular tropism, infecting only surface squamous epithelia of the skin or mucosa and producing for the most part benign epithelial tumors. Specific viral types appear to have a preference for either cutaneous or mucosal types. For example, HPV-11 does not readily infect cutaneous epithelium from other body sites but can infect mucosal epithelium of either the genital or the respiratory tract.

Genital warts are usually caused by HPV. Papilloma viruses cause small growths (warts) on the skin and mucous membranes. Infection of the genital and anal regions with HPV can cause warts (anogenital condyloma) on the penis, vulva, urethra, vagina, cervix, and around the anus (perianal). More than fifty different types of HPV have been classified. Several types, including 6 and 11, are associated with raised, rough, easily visible genital warts (especially in women). Other types are associated with flat warts. More importantly, several types are associated with pre-malignant and malignant changes in the cervix (abnormal Pap smears). These include types 16, 18, 31, 39, 45, 51, and 52. Research also shows that the presence of both HPV and herpes virus together is a good predictor of the development of cervical cancer.

Lesions on the external genitalia are easily recognized. On the penis, genital warts tend to be drier and more limited than on the female genitalia or around the anus of either sex. They grow best in the moist genital area, and are raised, rough, flesh-colored “warty” appearing tumors that may occur singly or in clusters. Left untreated, warts around the anus and vulva may rapidly enlarge, taking on a “cauliflower-like” appearance. In women, HPV can invade the vagina and cervix. These warts are flat and not easily visible without special procedures. Because HPV can lead to pre-malignant changes in the cervix (cervical dysplasia), it is important that this condition be diagnosed and treated. Regular Pap smears are important for detecting HPV.

Papillomavirus: A lesion in which there is evidence of a papillomavirus infection, such as identification of an HPV associated with the lesion.

Pharmaceutical agent or drug: A chemical compound or composition capable of inducing a desired therapeutic or prophylactic effect when properly administered to a subject. Pharmaceutical agents include, but are not limited to, chemotherapeutic agents and anti-infective agents.

Pharmaceutically acceptable carriers: The pharmaceutically acceptable carriers useful in this invention are conventional. Remington's Pharmaceutical Sciences, by E. W. Martin, Mack Publishing Co., Easton, Pa., 19th Edition (1995), describes compositions and formulations suitable for pharmaceutical delivery of the fusion proteins herein disclosed.

In general, the nature of the carrier will depend on the particular mode of administration being employed. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Precancerous lesions: Precancerous skin lesions are those areas of the skin in which tissue shows a tendency to undergo neoplasia. Examples of precancerous lesions include epithelial and mucosal lesions. Particular types of precancerous lesions include actinic keratosis, including Bowenoid actinic keratosis, arsenical keratosis, Bowen's disease, viral keratosis, vulval intraepithelial neoplasia, leukoplakia, erythroplaquia of queyrat, moles (nevi) and warts, including anogenital warts, such as anal warts, penile warts, vulval warts, vaginal warts and cervical papillomas.

Primary tumor: An original site of tumor development, as contrasted to a metastatic lesion.

Resonance modulator: A compound that possesses resonating intramolecular dipole movements (or electrical densities) that allow it to electrostatically interact with biological environments. A resonance modulator is also characterized by the emission of oscillating frequency waves generated by the compound's intramolecular resonance. This resonance is believed to convey the electrical dispositions for interactions with cells of the immune system (such as dendritic cells) to up-regulate and/or enhance immunity. Resonance modulators are capable of attracting immune cells, concentrating them in a target region of the peripheral immune system adjacent the resonance modulator, and in some instances have a distant effect on circulating immune cells (such as immune cells in the peripheral blood and lymphoid tissue such as a lymph node or the spleen). Many resonance modulators have a crystalline structure.

Therapeutically effective dose: A dose sufficient to inhibit or prevent advancement, or to cause regression of a condition that is being treated (such as a wart), or which is capable of relieving symptoms caused by the condition, such as pain or induration.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” In addition, the materials, methods, and examples are illustrative only and not intended to be limiting.

### III. Descriptions of Several Embodiments

The methods disclosed herein concern the use of a hydrazone compound that is a convenient and effective treatment for body surface lesions, such as infectious, neoplastic or inflammatory lesions, for example warts. Although not wanting to be bound by theory, it is believed that the disclosed compounds act as resonance modulators to activate PTPs. In certain examples, the resonance modulator acts as an immunostimulant and/or as a coupling agent between the immune system and external monitors or modulators. In certain examples, the resonance modulators are believed to interact with PTPs, for example interacting with cellular components of the immune system (such as CD45+ receptor lymphocytes or dendritic cells) to promote maturation of immune cells, and recruitment of other cellular components of immunity in an immune response. The ability of resonance modulators to attract immune cells to the vicinity of the modulator also allows an immune response to be directed to a target region within or on the body (such as a wart or malignant lesion) where the immune response can induce regression of the lesion.

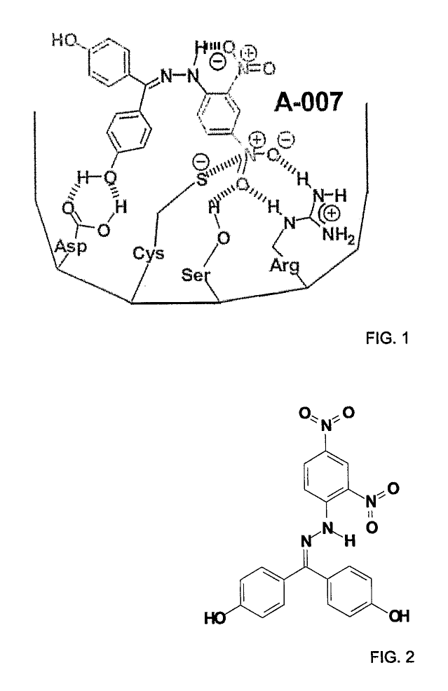

An example of a resonance modulator is 4,4′-dihydroxybenzophenone-2,4-dinitrophenylhydrazone, also known as A-007, the structure of which is illustrated in FIG. 2. This compound lacks substantial chemical reactivity, participates in no local chemical reactions, and has both an electronegative ground state and an affinity for cell membrane receptors. An example of its affinity for RPTP+ cell membrane receptors is the interaction between A-007 and the CD45+ T-lymphocyte surface receptor, which is illustrated in FIG. 1. These interactions are believed to induce maturation of the cells with which they interact, to promote the immune response.

### Example 1

### Treating Body Surface Lesions with Phenylhydrazones

A variety of compounds are disclosed in this example that are capable of treating body surface lesions such as warts and cancers, such as anogenital, genital, reproductive tract, or skin cancers. A particular suitable substance(s) having the desired therapeutic activity is a polyaryl mononitro- or dinitrophenylhydrazone such as

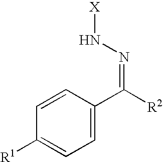

wherein R¹ is hydrogen, hydroxy, 2- or 4-hydroxyphenyl, acetate, phosphate, azido, nitrile, amino, dimethylamino, sulfate, methylsulfonate, phosphate, nitroso, succinate or another water soluble electrophilic group capable of hydrogen bonding; R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; and X is C₆H₃-2,4(NO₂)₂, C₆H₄-4(NO₂), C₆H₄-3(NO₂), or C₆H₃-2,4(NO₂)₂. In a particular example, R¹ is OH, R² is C₆H₄OH and X is C₆H₃-2,4(NO₂)₂.

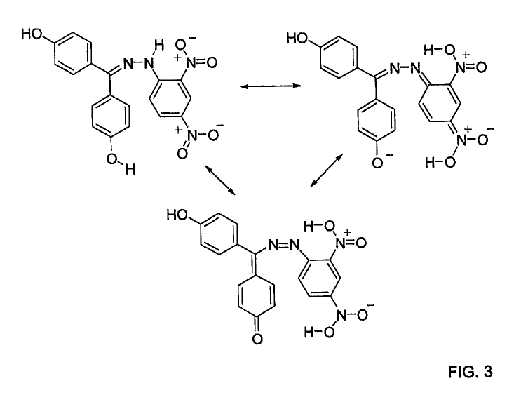

As described in great detail in WO 2004/078174 and illustrated in FIG. 3, these chemical structures are able to resonate through multiple dipolar configurations of conjugated electron rich/poor areas that allows/permits migrating intermolecular hydrogen bonding, as well as nucleophilic/electrophilic attractions with complementary sites to exist for moments in time. As illustrated in FIG. 1, these resonating mini-dipoles are capable of attracting for example —SH, —COOH, —NH₂—CH—NH₃⁺ that are present in amino acids (such as cysteine, threonine, arginine), carbohydrates (muccopolysaccharides) and glycoproteins, all of which are essential components of cellular regulators in the immune system. For example, simple interactions with 3 or 4 amino acids on the surface of the CD45+RA receptor on a lymphocyte is believed to induce the expression of a modified surface protein (RO, RB) capable of initiating the T-cell cascade with influxes of CD4+/CD8+ into tissues, organs and the circulation. In the “moment in time” during which the hydrogen bonding and attractions are modulated via electrical stimulation, the transient electrostatic interactions that intrinsically exist with these structures are increased and can be quantitated with NMR spectroscopy.

In a particularly disclosed embodiment of the hydrazone, R¹ is OH, R² is C₆H₄OH and X is C₆H₃-2,4(NO₂)₂, which is 4,4′-dihydroxybenzophenone-2,4-dinitrophenylhydrazone (A-007). This compound is highly electronegative, and exists in several resonance forms, as illustrated in FIG. 3.

### Example 2

### Additional Examples of Agents for the Treatment of Body Surface Lesions

A variety of other compounds disclosed herein are useful in the treatment of the body surface lesions, for example, compounds of the formula

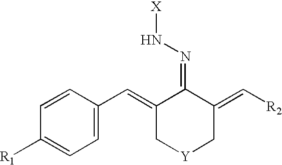

wherein R¹ is hydrogen, hydroxyl, 2- or 4-hydroxyphenyl, acetate, phosphate, azido, nitrile, amino, dimethylamino, sulfate, methylsulfonate, phosphate, succinate or another water soluble electrophilic group capable of hydrogen bonding; R₂ is C₆H₅, C₆H₄OH, C₆H₅, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; X is C₆H₃-2,4(NO₂)₂, C₆H₄-4(NO₂), C₆H₄-3(NO₂), or C₆H₃-2,4(NO₂)₂; and Y is —O—, —S—, —CH₂—, —N—, —, —CHA- or —CHOA-, wherein A is aryl, ester, amide, lipid, carbohydrate, or peptide residues. In a particular example, R¹ is OH, R² is C₆H₄OH, and X is C₆H₃-2,4(NO₂)₂.

In another example, the therapeutic agents have the structure:

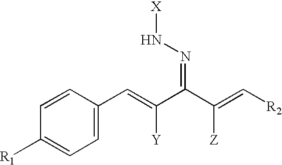

wherein R₁ is hydrogen, hydroxyl, 2- or 4-hydroxyphenyl, acetate, phosphate, azido, nitrile, amino, dimethylamino, sulfate, methylsulfonate, phosphate, succinate or another water soluble electrophilic group capable of hydrogen bonding; R₂ is C₆H₄OH, C₆H₄N₃, C₆H₄CN, CH₃, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; X is C₆H₃-2,4(NO₂)₂, C₆H₄-4(NO₂), C₆H₄-3(NO₂), or C₆H₃-2,4(NO₂)₂; Y is H, (CH)ₓCH₃ (x=0-12), —S—CH₃, nitrile, amino, nitro, azido, succinate, or amide; and Z is H, (CH)ₓCH₃ (x=0-12), —S—CH₃, nitrile, amino, nitro, azido, succinate, or amide. In a particular example, R₁ is H or OH, R₂ is C₆H₄OH or C₆H₅ and X is C₆H₃-2,4(NO₂)₂.

Other examples of therapeutic agents useful in the present method are shown in FIG. 12 of WO 2004/078174 (that Figure is incorporated herein by reference), and include formyl and acetylbarbituric phenylhydrazone analogs (analogs 15-21), formylbarbituric acid Schiff base analogs (analogs 22-25), as well as the other resonance modulators shown as compounds 26-37 in WO 2004/078174. Compound 26, for example, is an anthracene. These and other compounds are believed to have immune modulation properties, as well as electromagnetic characteristics that enable them to function as quantum sensors.

The structures and synthetic methods for making many of the described compounds have been previously described in U.S. Pat. No. 4,732,904, which structures and methods are incorporated by reference. Some compounds that were not described in that patent include 10-[(2,4-dinitrophenyl)hydrazono]-1,2-dihydroxy-10H-anthracen-9-one (DNPA). The synthesis of this compound was performed by dissolving 0.1 mole of 1,2-dihydroxy-9,10-anthraquinone in 50 ml of ethanol and 5 ml sulfuric acid. Gently the solution was heated to 60° C. with stirring and 0.1 mole of 2,4-dinitrophenylhydrazone in 50 ml of ethanol was added slowly with continued stirring. The solution was heated for 2-hrs and cooled in a refrigerator. Deep red crystals of DNPA appeared; m.p. 240-242° C.; yield 92%. The product analyzed for C₂₀H₁₂N₄O₇—found: C, 63.98; H, 3.46; N, 11.13; NMR and mass spectra agreement.

### Example 3

### Preparation of Pellets and Gels

The agents for treating body surface lesions can be provided in many forms, such as a crystal, pellet or gelcream. The agent can be, for example, implanted subcutaneously or applied to a body surface lesion being treated. The active agent can be incorporated into any form (such as a patch, or a two-dimensional or three-dimensional matrix) that brings it into contact with a target site. In some examples, the agent is applied to or over a lesion (such as a wart or cutaneous cancer) that is being treated. The active agents can also be chemically modified if desired, for example to form polymers.

In one example, a pellet of A-007 was prepared by pressing it from 50 mg of pure chemical and sizing it to 16-gauge. Bulk A-007 was prepared using GLP/GMP procedures, with no additives. Depending upon the dissolution properties of the 100% A-007 pellet, additives (stearic acid, povidone, etc) or other pharmaceutically acceptable carriers may be added to it. Pellets may be manufactured, for example, in 25 mg, 50 mg and 75 mg doses. Adjustments in pellet concentrations may be made according to the observed physical properties (such as dissolution rates) and animal toxicity. Therapeutically effective doses can be determined by known means, and doses to be administered can be varied depending on the condition being treated, or the severity of a disease.

The active agents can also be provided in the form of a gel, such as a preparation of propylene glycol/methyl cellulose, for application to a target area. The gels may be provided in a tube, such as 50 mg of the active agent suspended in the gel. These gels are particularly convenient for application to the skin or other epithelial surface (for example skin of the foot, face or anogenital area, or cervical mucosa). In some embodiments, the gel is dispensed from an elongated applicator tube (such as a rectal or vaginal applicator), for example to apply it rectally or intravaginally.

Alternatively, a pellet can be inserted into a target region, such as a wart. In other examples, the agent is suspended in tetrahydrofuran (THF) or another non-toxic aerosol, and introduced into to the tracheobronchial or oropharyngeal area to treat body surface infections of the oral cavity or upper respiratory tract.

### Example 4

### Treatment of Plantar Warts

A 66-year old female had a long history of plantar warts accompanied by increasing induration on her left foot. Prior attempts at local surgical removal of the warts had failed. She had recently had a deep excision of the wart with freezing which provided partial relief, but after two weeks the induration returned. A-007 was applied in a 0.25% gel (2 g) nightly for three nights and covered with an absorbent pad to allow maximum penetration. After 7 days the area began peeling and new skin appeared which was soft with no area of induration. Neither the warts nor induration returned after two months of follow-up observation.

### Example 5

### Treatment of Cervical Warts

A 33-year old female had multiple abnormal pap smears with hair loss and skin changes secondary to hormonal imbalance. She was found to have CIN II, as well as high risk HPV infection and cervical warts. She was treated with 2 grams per day of 0.25% gel applied to the vagina for five days, for a total topical dose of 10 grams. No toxicities were observed, her CIN resolved, and the warty lesions on her cervix disappeared. The HPV infection converted to low grade, and she was stable after 18 months of observation.

### Example 6

### Treatment of Condyloma

A 35-year old female had a condyloma on her cervix accompanied by warty dysplasia compatible with HPV infection. A review of the HPV by hybrid capture revealed that she had a low risk HPV in the 6/11 category. She also had atypical squamous epithelium and CIN with warts. The cervix was inflamed with squamous exo-cervical dysplasia and a warty labial growth. She was treated with 2 grams per day of 0.25% A-007 gel applied to the vagina for five days, for a total topical dose of 10 grams. The cervical condyloma disappeared and the external warty mass fell off the labia. The HPV infection was also eliminated, and was absent after 24 months of observation.

Although shown as a gel in this example, the A-007 could also be applied as a cream or simple crystals (50 mg) or a pellet (50-100 mg) that can be applied on to or inserted into the epithelial surface or the skin (or other surface) with a 16-gauge trochar needle for short-term treatments.

### Example 7

### Treatment of Penile Cancer

A subject is diagnosed with a primary penis cancer. The subject is then administered A-007 on the surface of the lesion in a 0.25% gel, in a dose of 2 g per day, until the lesion is resolved. In some instances the agent is topically applied for 3-10 days for a total dose of 6-20 grams. This regimen may be repeated if necessary unless desired regression or resolution of the lesion is achieved. It is expected that no further occurrences of the lesion would develop, but if the lesion recurs, the treatment can be repeated. Because of its relative lack of toxicity the agent can be repeatedly applied over long periods of time, for example for many weeks or months.

### Example 8

### Treatment of Vulval Cancer

A subject is diagnosed with Stage I squamous vulval cancer. The subject is administered A-007 on the surface of the tumor in a 0.25% gel, in a dose of 2 g per day, until the tumor is resolved. In some examples the agent is applied for 3-10 days for a total dose of 6-20 grams. Optionally, the treatment can be repeated, with or without a period of no treatment of, for example from 1 to 25 days. The treatment can be repeated as necessary until the tumor has completely disappeared. It is expected that the vulval tumor would not recur, but recurrences can be treated with A-007 or another therapeutic compound disclosed herein having anti-tumor activity.

### Example 9

### Treatment of Other Body Surface Lesions

The agents disclosed herein (for example A-007) can be used in the treatment of a broad variety of body surface lesions, such as warts, HSV blisters, psoriasis and skin cancers. These lesions are treated by applying the agent topically to the lesion for a sufficient period of time to induce disappearance or regression of the lesions. For example, the agent is applied to the surface of the lesion in a 0.25% gel, in a dose of 2 g per day, for 3-10 days. The treatment can be repeated as needed until resolution of the lesion has been achieved.

Other treatment regimens can be used. For example, a higher or lower dosage of the active ingredient can be used as clinically appropriate. Therapeutically effective amounts of the drug can be determined by clinicians depending on the clinical circumstances. For example, a total dose of 1-10 g, 2-5 g, or 2-3 g can be administered topically during a treatment regimen. Dosage regimens can be once a day (for example nightly), twice a day, or more often. The total duration of treatment can vary depending on the clinical circumstances (for example daily for 3-10 days, for example daily for 5 days), and the treatment regimens can be repeated at intervals as necessary. For example, treatment can be administered daily for 5 days followed by 25 days of no treatment followed by another course of daily treatment for 5 days.

### Example 10

### Further Examples of Agents Useful in Treating the Body Surface Lesions

Another example of an agent that can be used in the treatment of the body surface lesions includes 2,6-dibenzylidenecyclohexanone-2,4-dinitrophenylhydrazone (BDP-DNP).

Yet other examples of effective agents useful in treating the body surface lesions can be found in co-pending provisional application 60/678,089. The disclosure of those compounds and the methods of making them are incorporated by reference. Some of these agents include compounds having the formula

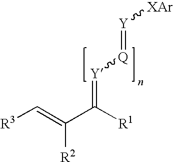

wherein Ar comprises an aryl group substituted with at least one electron withdrawing group;

X is NH, CH₂, O, —, SO₂, or B;

Q is —(CH═CH)ₘ—CH—;

Y and Y′ independently are selected from N, CH and B;

n is 0 or 1;

m is from 0 to 6;

R¹ is H, lower alkyl, optionally substituted phenyl or

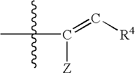

wherein R⁴ is an optionally substituted phenyl group;

R² is optionally substituted lower alkyl, aralkyl, thioalkyl, amidoalkyl, amino, amino alkyl, nitro, azido; together with Z forms an optionally substituted 4-7 membered carbocyclic or heterocyclic ring; or together with R⁴ forms an optionally substituted phenyl group;

R³ with R² forms an optionally substituted phenyl group or is an optionally substituted phenyl group; and

Z is H, lower alkyl, substituted alkyl, thioalkyl, nitrile, amino, or together with R² forms an optionally substituted 4-7 membered carbocyclic or heterocyclic ring.

In one embodiment, the compounds are derivatized with a biomolecule, such as a carbohydrate, amino acid, peptide, fatty acid, nucleoside or nucleic acids. In one aspect, such derivatization is used to enhance a compound's solubility and/or target a compound to a particular tissue, cell or receptor. In one embodiment, an optionally substituted phenyl group, as recited with respect to the general formula above, comprises such a biomolecule.

In certain embodiments, disclosed compounds include, for example, compounds having the formula above; wherein n is 0, such as compounds of the formula

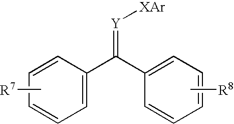

wherein R⁷ and R⁸ independently are —OR⁵, —SR⁵, —SO₂N(R⁵)₂, —SO₂CN, —SO₂R⁶, —SO₂N₃, N₃, —CN, —N(R⁵)C(O)R⁶, —R⁶;

each R⁵ independently is H, lower alkyl, carbohydrate, fatty acid, amino acid, peptide, nucleoside or nucleic acid; R⁶ is lower alkyl or C-glycoside; and Ar, X and Y are as described above.

In another embodiment, X represents a bond. Exemplary structures wherein X represents a bond include

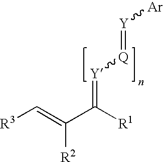

Exemplary embodiments of such compounds, when n=0, are represented by the formula

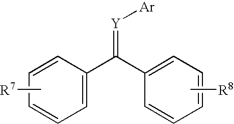

wherein Ar, Y, R⁷ and R⁸ are as defined above.

In one embodiment, the compounds have the formula

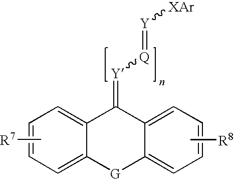

wherein G represents —CH₂—, —C(O)—, —N—, —, —O—, or —S—; n is 0 or 1; and Y′, Q, Y, X, Ar, R⁷ and R⁸ are as set forth above.

Other compounds disclosed herein include those having the formula

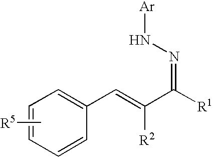

wherein R⁵ is H, —OR⁶, —SR⁶, —SO₂N(R⁶)₂, —SO₂CN, —SO₂R⁷, —SO₂N₃, N₃, —CN, —N(R⁶)C(O)R⁷, or —R⁷;

R¹ and R⁶ independently are H or lower alkyl;

R² and R⁷ independently are lower alkyl; and Ar is an aryl group as set forth above.

Certain embodiments of compounds disclosed herein are illustrated by the following formula:

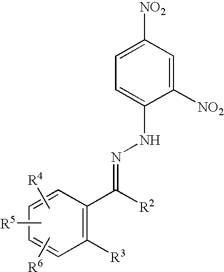

wherein R² is H, lower alkyl or optionally substituted phenyl; R³ is H, or together with R² forms an optionally substituted heterocyclic or carbocyclic ring, which optionally is fused to an aryl group; R⁴, R⁵ and R⁶ independently are selected from H, —OH and —OR⁷.

Particular embodiments of compounds disclosed herein include arylhydrazones. Other examples include aryl nitrohydrazones, such as phenylhydrazones, such as polyaryl mononitro- or dinitrophenylhydrazones, for example

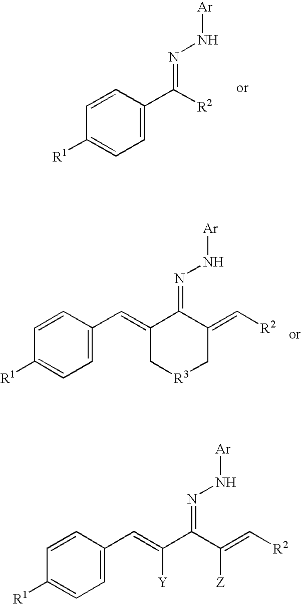

wherein R¹ is hydrogen, hydroxy, hydroxyphenyl (such as 2- or 4-hydroxyphenyl), acetate, phosphate, azido, nitrile, amino, dimethylamino, sulfate, methylsulfonate, phosphate, succinate;

R² is an unsubstituted (C₆H₅) or substituted phenyl group such C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;

Ar is nitrophenyl, such as C₆H₃-2,4(NO₂)₂, C₆H₄-4(NO₂), C₆H₄-3(NO₂), or C₆H₃-2,4(NO₂)₂;

R³=—O—, —S—, —CH₂—, —N—, —, —CHA- and —CHOA-; where A=aryl, ester, amide, lipid, carbohydrate, or peptide;

Y=H, (CH)ₓCH₃ (x=0-12), —S—CH₃, nitrile, amino, nitro, azido, succinate, or amide; and

Z=H, (CH)ₓCH₃ (x=0-12), —S—CH₃, nitrile, amino, nitro, azido, succinate, or amide.

### Example 11

### General Procedure for Preparation of Hydrazones

General Method 1: Substituted phenylhydrazines (0.148 mol) were suspended in MeOH (300 mL) at 50° C. and concentrated sulfuric acid (20 mL) added with stirring at 50° C. After the hydrazine dissolved, the substituted the substituted benzophenone or aryl ketone (0.1 mol) in MeOH (300 mL) was added with stirring at 50° C. over 10 minutes and then stirred for another 30 min. The solvent was evaporated (75%), water (500 mL) added, and the precipitate washed with 3% aqueous NaHCO₃. Recrystallizations were from EtOH, MeOH, or acetic acid. Complete elemental and spectral analysis were conducted on all products.

General Method 2: A second general method proceeds as follows: Substituted aryl hydrazines (2.7 mmol) were dissolved in 15 mL MeOH and 2.1 mmol of the ketone added with stirring. Dowex 50W-50X2-100 cation exchange resin (0.5 g) [Dow Corp., Baton Rouge, La.] was added. The suspension was refluxed for 1 h at 75° C. on a water bath. The mixture was filtered hot under water vacuum pressure and the resin washed with 3×0.5 mL of MeOH. The contents of the collection flask were allowed to cool to room temperature and 20 mL of distilled water added. The colored precipitates were filtered, dried and recrystallized from EtOH or MeOH. Representative yields were in the 80% range.

N-Benzhydrylidene-N′-(2,4-dinitrophenyl)hydrazine was prepared using method 1, yielding yellow crystals in 82% recovery from EtOH or MeOH; mp 232-234° C.; IR: 3295 (N—H), 2928 (Ar—H), 1612 (C═N), 1590, 1504, 1416, 1334, 1127 and 1091, 834, 777, 698 and 608 cm⁻¹; ¹H-NMR (DMSO, 400 MHz): [symbol] 11.25 (1H, s, N—H), 9.07 (1H, s), 8.37 (1H, d, J=8 Hz), 8.22 (1H, d, J=8 Hz), 7.67 (4H, m, J=8 Hz) 7.41 (6H, m, J=8 Hz); Anal. Cald'd for C₁₉H₁₄N₄O₄: C, 62.98; H, 3.86; N, 15.46. Found: C, 63.17; H, 4.06; N, 15.49.

4-Hydroxybenzophenone-2,4-dinitrophenylhydrazone was prepared using method 2, and was obtained as red needles that recrystallized from ETOH—yield 80%; mp 224-225° C.; IR: 3541 (N—H), 3271 (N—H), 3098 (O—H), 2905 (Ar—H), 1621 (C═N), 1591, 1511, 1418, 1329, 1276, 1130, 1083, 829 and 698 cm⁻¹; ¹H-NMR (DMSO, 400 MHz): [symbol] 11.23 (½H, s, N—H), 10.99 (½H, s, N—H), 10.1 (1H, s, “broad” OH), 8.8 (1H, s), 8.41 (1H, d, J=8 Hz), 8.19 (1H, d, J=8 Hz), 7.67 (2H, m, J=8 Hz), 7.48 (3H, m, J=8 Hz), 7.28 (1H, dd, J=8 Hz), 7.04 (1H, dd, J=8 Hz), 6.84 (1H, dd, J=8 Hz); Anal. Calc'd. for C₁₉H₁₄N₄O₅: C, 59.07; H, 3.62. Found: C, 59.71; H, 3.84.

2,2′,4,4′-Tetrahydroxybenzophenon-2,4-dinitrophenylhydrazone was synthesized as deep red crystals that recrystallized from EtOH—yield 75%; mp 290-291° C.; IR: 3631 (N—H), 3491 (N—H), 3272 (O—H), 2905 (Ar—H), 1614 (C═N), 1518, 1415, 1322, 1206, 1143, 1107, 974, 864 and 827 cm⁻¹; ¹H-NMR (DMSO, 400 MHz): δ 11.59 (1H, s, O—H), 11.15 (1H, s, N—H), 10.12 (2H, s, O—H), 9.91 (1H, s, O—H), 8.85 (1H, s), 8.48 (1H, d, J=8 Hz), 8.19 (1H, d, J=8 Hz), 7.61 (1H, d, J=8 Hz), 6.95 (1H, dd, J=8 Hz), 6.8 (1H, dd, J=8 Hz), 6.57 (1H, s), 6.46 (1H, dd, J=8 Hz), 6.38 (1H, s), 6.31 (1H, dd, J=8 Hz); Anal. Calc'd. for C₁₉H₁₄N₄O₈: C, 53.52; H, 3.28; N, 13.14. Found: C, 51.79; H, 3.19; N, 13.36.

1,2-Dihydroxy-10-anthraquinone-2,4-dinitrophenylhydrazone was synthesized from the base quinone and 2,4-DNP using General Method 1. Yield was 92%. The reaction mixture was refluxed for 48 h. The compound precipitated from the reaction upon cooling and recrystallized from dioxane/hexane as red microcrystals; mp 240-242° C.; IR: 3502 (N—H), 3287 (N—H), 3113 (O—H), 2925 (Ar—H), 2559 (Ar—H), 1618 (C═N), 1595, 1501, 1441, 1335, 1298, 1135, 1091 and 781 cm⁻¹; ¹H-NMR (DMSO, 300 MHz): δ 11.97 (1H, s, N—H), 8.86 (1H, s), 8.47 (1H, d, J=6), 8.38 (1H, s), 8.24 (1H, d, J=12 Hz), 8.17 (2H, m), 8.12 (2H, m), 7.89 (4H, m), 7.75 (3H, m).

### Example 12

### Treatment of Psoriasis

This example addresses the use of the disclosed agents in the treatment of psoriasis. Any of the disclosed agents (for example A-007) are believed to be useful for this purpose, but especially suitable compounds will be those with extra double bonds attached to the —C═N moiety. Specific examples of such compounds include, without limitation, those having the formula

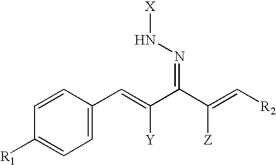

wherein R₁ is hydrogen, hydroxyl, 2- or 4-hydroxyphenyl, acetate, phosphate, azido, nitrile, amino, dimethylamino, sulfate, methylsulfonate, phosphate, succinate or another water soluble electrophilic group capable of hydrogen bonding; R₂ is C₆H₄OH, C₆H₄N₃, C₆H₄CN, CH₃, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl; X is C₆H₃-2,4(NO₂)₂, C₆H₄-4(NO₂), C₆H₄-3(NO₂), or C₆H₃-2,4-(NO₂)₂; Y is H, (CH)ₓCH₃ (x=0-12), —S—CH₃, nitrile, amino, nitro, azido, succinate, or amide; and Z is H, (CH)ₓCH₃ (x=0-12), —S—CH₃, nitrile, amino, nitro, azido, succinate, or amide. In a particular example, R₁ is H or OH, R₂ is C₆H₄OH or C₆H₅ and X is C₆H₃-2,4(NO₂)₂.

For the treatment of psoriasis, an effective amount of the compound is applied to psoriasis lesions on the skin, for example once a day for 3-5 days. This treatment regimen is repeated, for example by repeating this course every 3-6 weeks, or when the lesions recur or worsen in severity.

Having illustrated and described the principles of the invention in several examples, it should be apparent to those skilled in the art that the invention can be modified in specific details without departing from such principles. I claim all modifications coming within the spirit and scope of the following claims.

I claim:

1. A method of treating a wart, comprising applying to the wart an effective amount of a compound comprising 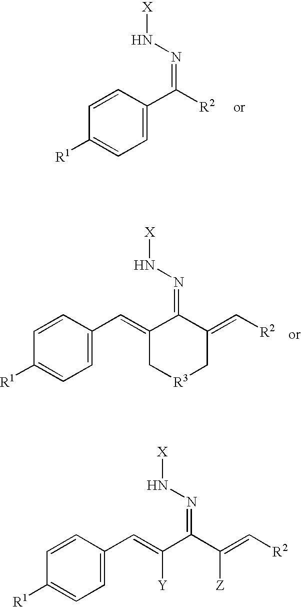
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³=—O—, —S—, —CH₂—, —N—, —, —CHA- and —CHOA-; where A=aryl, ester, amide, lipid, carbohydrate, or peptide;
Y=H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

2. The method of claim 1, wherein the wart is a non-venereal wart.

3. The method of claim 2, wherein the wart is a plantar wart, filiform wart, flat wart or seborrheic wart.

4. The method of claim 1, wherein the wart is an anogenital wart.

5. The method of claim 4, wherein the wart is an anal, cervical, oral, penile, vaginal or vulval wart.

6. The method of claim 4, wherein the wart is a papilloma virus associated wart.

7. The method of claim 6, wherein the wart is an HPV-induced wart.

8. The method of claim 1, wherein the wart is an external body surface wart.

9. The method of claim 1, wherein the wart is an internal body surface wart.

10. The method of claim 9, wherein the wart is a cervical wart.

11. The method of claim 1, wherein treating the wart comprises providing the compound in a topical pharmaceutical carrier and applying the compound topically to the wart.

12. The method of claim 1, wherein treating the wart comprises providing the compound in a pellet that is introduced into or adjacent the wart.

13. The method of claim 11, wherein the wart is a plantar wart.

14. The method of claim 1, wherein the compound is 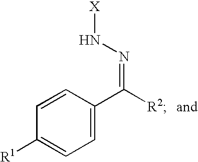
R¹; R² and X are as set forth in claim 1.

15. The method of claim 1, wherein the compound is 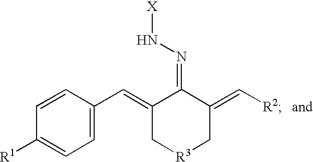
R¹; R²; R³; and X are as set forth in claim 1.

16. The method of claim 1, wherein the compound is 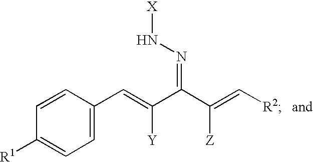
R¹; R²; R³; X; Y and Z are as set forth in claim 1.

17. The method of claim 1, wherein R¹ is OH, R² is C₆H₄OH and X is C₆H₃-2,4(NO₂)₂.

18. The method of claim 14, wherein the compound is 2,6-dibenzylidenecyclohexanone-2,4-dinitrophenylhydrazone.

19. A method of treating a hyperproliferative body surface lesion, comprising applying to the lesion an effective amount of a compound comprising 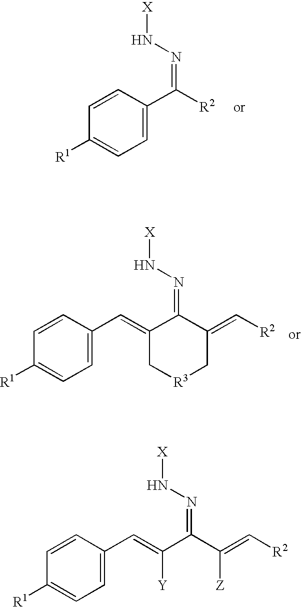
wherein R¹ is hydrogen, hydroxy, 2- or 4-hydroxyphenyl, acetate, phosphate, azido, nitrile, amino, dimethylamino, sulfate, methylsulfonate, phosphate, succinate;
R² is C₆H₅, C₆H₄OH, C₆H₄N₃, C₆H₄CN, 4-HO—C₆H₄—C₆H₄, C₆H₄OPO₂OH, C₆H₄OSO₂H, C₆H₄NH₂, C₆H₄NHMe₂, C₆H₄OSO₂Me, C₆H₄OCO(CH₂)ₓCO₂H, or C₆H₅Cl;
X is C₆H₃-2,4(NO₂)₂, C₆H₄-4(NO₂), C₆H₄-3(NO₂), or C₆H₃-2,4(NO₂)₂;
R³=—O—, —S—, —CH₂—, —N—, —, —CHA- and —CHOA-; where A=aryl, ester, amide, lipid, carbohydrate, or peptide;
Y=H, (CH)ₓCH₃ (x=0-12), —S—CH₃, nitrile, amino, nitro, azido, succinate, or amide; and
Z=H, (CH)ₓCH₃ (x=0-12), —S—CH₃, nitrile, amino, nitro, azido, succinate, or amide.

20. The method of claim 19, wherein the hyperproliferative body surface lesion is a precancerous lesion.

21. The method of claim 20, wherein the lesion is an anal, cervical, oral, penile, vaginal or vulval lesion.

22. The method of claim 20, wherein the precancerous lesion comprises actinic keratosis, Bowenoid actinic keratosis, arsenical keratosis, Bowen's disease, viral keratosis, leukoplakia, erythroplaquia of queyrat, a nevus or a wart.

23. The method of claim 20, wherein the precancerous lesion is papilloma virus associated lesion.

24. The method of claim 19, wherein the hyperproliferative body surface lesion is a primary neoplastic lesion.

25. The method of claim 24, wherein the neoplastic lesion is anal cancer, cervical cancer, basal or squamous cell carcinoma, melanoma, penile cancer, vulval cancer, vaginal cancer or cancer of the oral mucosa.

26. The method of claim 19, wherein the hyperproliferative body surface lesion is a metastatic neoplastic lesion.

27. The method of claim 19, wherein the lesion is a penile lesion.

28. The method of claim 19, wherein the lesion is a vulval or vaginal lesion.

29. The method of claim 19, wherein the body surface lesion is an internal body surface lesion.

30. The method of claim 19, wherein the body surface lesion is an external body surface lesion.

31. The method of claim 19, wherein the body surface lesion is an anogenital lesion.

32. The method of claim 31, wherein the anogenital lesion is a genital lesion.

33. The method of claim 32, wherein the genital lesion is a genital wart.

34. The method of claim 32, wherein the genital lesion is a lesion of a female reproductive tract.

35. The method of claim 32, wherein the genital lesion is a lesion of a cervix.

36. The method of claim 31, wherein the anogenital lesion is an anal wart.

37. The method of claim 19, wherein the lesion is a papillomavirus associated lesion.

38. The method of claim 37, wherein the papillomavirus associated lesion is an HPV-induced lesion.

39. The method of claim 19, wherein applying the compound to the lesion comprises topically applying the compound to the body surface lesion.

40. The method of claim 19, wherein applying the compound to the body surface lesion comprises introducing the compound into the skin.

41. The method of claim 19, wherein treating the body surface lesion comprises treating a skin lesion induced by infection or inflammation.

42. The method of claim 41, wherein treating the body surface lesion comprises treating a skin lesion induced by infection.

43. The method of claim 42, wherein treating the body surface lesion comprises treating a skin lesion induced by a virus.

44. The method of claim 43, wherein treating the body surface lesion comprises treating a virally induced wart.

45. The method of claim 44, wherein treating the body surface lesion comprises treating a papillomavirus induced wart.

46. The method of claim 44, wherein treating the body surface lesion comprises treating a herpes virus infection of the skin.

47. The method of claim 19, wherein treating the body surface lesion comprises treating a skin neoplasm.

48. The method of claim 19, wherein treating the body surface lesion comprises treating a psoriasis lesion.

49. The method of claim 48, wherein treating the psoriasis lesion comprises applying to a psoriasis lesion the compound

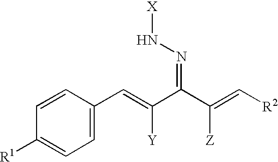

50. A method for the prophylaxis of a body surface cancer, comprising contacting a precancerous lesion with an effective amount of 4,4′-dihydroxybenzophenone-2,4-dinitrophenylhydrazone.

51. A method for treating a body surface cancer selected from anal, cervical, penile, vulval or vaginal cancer, comprising contacting the body surface cancer with an effective amount of 4,4′-dihydroxybenzophenone-2,4-dinitrophenylhydrazone.

52. The method of claim 51, wherein the cancer is a primary tumor.

53. The method of claim 51, wherein the cancer is a metastatic cancer.

54. The method of claim 51, wherein the cancer is cervical cancer.

55. The method of claim 51, wherein the cancer is anal cancer.

56. The method of claim 51, wherein the cancer is penile cancer.

57. The method of claim 51, wherein the cancer is vulval or vaginal cancer.
